(19) United States
(12) Patent Application Publication
(10) Pub. No.: US 2013/0118590 A1
(43) Pub. Date: May 16, 2013

(54) METHOD FOR REDUCING COSTS OF ENZYMES IN BIOREFINERY

(71) Applicants: Andrew Desbarats, Aurora (CA); Michael Erhart, Phillipsburg, KS (US); Vince Ycyshyn, Calgary (CA)

(72) Inventors: Andrew Desbarats, Aurora (CA); Michael Erhart, Phillipsburg, KS (US); Vince Ycyshyn, Calgary (CA)

(73) Assignee: SCIENTECK LLC, Phillipsburg, KS (US)

(21) Appl. No.: 13/810,748

(22) PCT Filed: Jul. 18, 2011
(86) PCT No.: PCT/US11/44307

§ 371 Date: Jan. 17, 2013

Related U.S. Application Data
(60) Provisional application No. 61/443,954, filed on Feb. 17, 2011.
(60) Provisional application No. 61/365,905, filed on Jul. 20, 2010.

(51) Int. Cl. C12N 9/00

(52) U.S. Cl. CPC C12N 9/00
USPC 137/1

(57) ABSTRACT

A business methodology for optimizing enzyme use rates and enzyme supply and reducing greenhouse gas emissions for an industrial process that employs enzymes as part of the production process by changing the concentration, specific gravity and/or activity of an enzyme prior to, and just-in-time for, the addition of said enzyme to a reactor wherein an enzyme-catalyzed reaction occurs. After collecting data that describes the extent to which enzyme consumption has decreased as a result of the change in concentration, specific gravity and/or activity of said enzymes, the enzyme user can pay to the provider of technology that allows enzyme consumption to decrease, a proportion of the savings.

This application claims priority to U.S. Provisional Patent Application Ser. Nos. 61/365,905, filed 20 Jul. 2010, and 61/443,954, filed 17 Feb. 2011, the complete disclosures of which are incorporated herein by reference.

### FIELD OF THE INVENTION

This invention relates to a process for the efficient dosing of industrial enzymes into an industrial process. The results of the invention include reduced shipping costs for enzyme manufacturers and enzyme users, and reduced enzyme costs for enzyme users by enabling enzyme users to fine-tune the enzyme dose for their particular application.

### BACKGROUND OF THE INVENTION

Commercial enzyme formulations are provided to industrial enzyme users such as high fructose corn syrup (HFCS) manufacturers, fuel ethanol producers, bakeries, breweries and others in the form of a standard concentration, activity and specific gravity. Processes that use enzymes differ across, and even within, industries in terms of rate of enzyme addition to the process. However enzyme formulations are often standardized to reduce production costs for enzyme producers. In most cases these commercial enzyme formulations are dosed into the manufacturing process in a continuous mode using pumps and flow meters to control dosing. Currently, the rate of addition is controlled by the enzyme user, but the initial concentration, specific gravity and/or activity of the enzyme dosed to the process cannot be controlled; they are based on what the supplier provides.

It is well documented that the initial concentration of enzyme formulations can be changed prior to addition to a reactor wherein an enzyme-catalyzed reaction takes place. As early as 1924, John Northrop found that, when substrate is available in excess, the amount of product produced in an enzymatic reaction is proportional to the concentration of the enzyme used to produce said product. As the initial concentration of enzyme is reduced, the rate of reaction decreases. Similarly, many current enzyme product monographs from commercial enzyme companies indicate that users can reduce the enzyme concentration prior to use. In production systems where enzymes are added as a batch, changing the enzyme concentration is straightforward. However in processes where enzymes are added continuously, changing the concentration of these enzymes as they are being added to the production process is less obvious. It is easier for enzyme users to adjust the flow rate of concentrated enzymes. This only changes the final enzyme concentration of enzyme in the reactor, but does not change the initial enzyme concentration.

Merely reducing the volume of concentrated enzymes added to a substrate also affects diffusion of a small volume of enzyme relative to a large volume of substrate. This effect is especially important when said small volume of enzyme is added drop-wise to a large volume of substrate that is pumped at high flow rates through large pipes to a bioreactor. In many industrial applications such as those listed above, the substrate is a viscous slurry containing between 10 and 40% solids. The high viscosity common to many biorefinery liquids exacerbates this problem of diffusion of concentrated enzymes. A dropwise addition of concentrated enzyme may enable the enzyme-substrate reaction in the region around which the enzyme drop was received into the substrate slurry, however diffusion may be limited. The result is channelling in the substrate slurry and incomplete conversion of substrate to product.

Additional problems exist in adjusting the concentration of commercial enzyme preparations. Preservatives such as glucose, sucrose, glycerol, methionine, salts and others are added to commercial enzyme formulations to prevent bacterial and fungal growth and to stabilize the enzymes so that they remain active over long periods of time, often up to one year. Reducing the enzyme concentration also results in a reduction of preservative concentration. Bacterial growth will occur quickly in an enzyme solution when the preservative concentration is reduced significantly. Therefore, the careful timing of enzyme concentration, activity and/or specific gravity adjustment and use in an enzyme-catalyzed reaction must be tightly controlled to prevent the introduction of bacterial and fungal contamination into the reactor where the enzyme-catalyzed reaction takes place.

### SUMMARY OF THE INVENTION

It has been found through a substantial amount of testing at the commercial scale, that the ability to adjust enzyme concentrations in a just-in-time manner in a continuous process provides unexpected benefits such as fine-tuning the enzyme concentration, specific gravity and/or activity to an extent that provides more accuracy than simply changing the flow rate of commercial enzyme that comes as a standard concentration, specific gravity and/or activity. Fine-tuning the dose to the extent possible through just-in-time processing can reduce the amount of enzyme required in an industrial process thus reducing enzyme-related costs for enzyme users. In addition, greater accuracy in enzyme dosing can improve the yields in the enzyme-catalyzed reaction thereby increasing the profitability of the enzyme user. This is especially true where enzyme is added in small amounts to a fast flowing stream of a slurry based substrate. Finally, the present method wherein enzyme concentration, specific gravity and/or activity, can be changed on a just-in-time basis ensures that the reformulated enzyme solution is pumped to the desired reactor before harmful quantities of bacteria and fungus are allowed to accumulate.

An additional unexpected benefit of the present method of administering enzymes is directed at enzyme producers. Whereas in the past, enzyme concentration was limited by slower rates of diffusion in slurry systems and lower pumpability of viscous, concentrated enzyme solutions, enzyme producers can use the present invention to supply more concentrated enzyme solutions to industrial users, who can then tailor the concentration, specific gravity and activity according to their process parameters. This results in less frequent shipments and lower emissions further reducing the cost of supplying enzymes. In addition, the present invention can free up capacity for producing more enzymes without investing in additional enzyme production equipment. Reduced emissions from fewer deliveries of enzyme, as the present invention provides, improves the energy balance of ethanol production. Reduced transportation fuel requirements for enzyme delivery reflects an improvement in the ratio of energy provided by ethanol compared to the energy input required to produce ethanol. In addition, the efficiencies that the present invention provides can be used as offset credits to offset greenhouse gas emissions. For example, fossil fuels consumed in the delivery of chemicals such as enzymes is classified as ‘Scope 2’ emissions. A reduction in these emissions will reduce the total emissions of ethanol plants and will reduce the cost that ethanol plants must incur either in the form of a carbon tax or in the purchase of emissions credits in a cap & trade emissions reduction scheme.

A particular unexpected advantage of the present method of administering enzymes to reactors applies to industries where large quantities of enzyme are required. One such industry is the conversion of non-food biomass to sugar for the production of ethanol, butanol or other biofuels. This industry requires frequent enzyme deliveries. Currently, enzymes are delivered in trailer loads, and frequent deliveries consume high quantities of transportation fuel. In addition to the high cost of transportation fuel to deliver enzymes from a central enzyme production location to distributed biorefineries, there will also be a large quantity of greenhouse gases and volatile compound emissions as a result of this practice. By providing the flexibility to adjust the enzyme concentration just-in-time, enzyme companies can now increase the concentration of the enzymes provided to these users and reduce shipment frequency which will result in reduced enzyme costs, reduced enzyme storage costs, reduced greenhouse gas emissions and reduced emissions of volatile compounds from the burning of fossil fuels and an improved net energy value of ethanol from biomass.

An additional unexpected benefit of the present method of administering enzymes is that an enzyme mixture can be tailored, on site, and just-in-time, that corresponds to the feedstock being hydrolyzed in the biorefinery. Biorefineries where cellulosic feedstocks are hydrolyzed to produce biofuels and other products, employ various feedstocks each of which may require a particular enzyme in a particular concentration. The present method allows operators to select the enzyme mixture and the concentrations, specific gravities and/or activities of each enzyme as different feedstock mixtures are used in the plant. This can alleviate time-consuming feedstock and enzyme changeovers, making biorefineries more efficient.

Users of industrial enzymes are often operating in industries where margins are low and profits are a function of commodity inputs and commodity outputs, leaving the user of industrial enzymes exposed to commodity price fluctuations. To relieve the industrial enzyme user of the need to acquire funding for the purchase of an enzyme reformulation unit, such as the one described in patent application WO/2010/045168, published 22 Apr. 2010, a savings sharing plan is also the subject of the present invention. The enzyme reformulation unit is leased to the enzyme user. The cost of the lease is subject to the amount of enzyme that is reformulated and the resulting cost savings. The lessor receives a portion of the savings and is only paid when the lessee is successfully reducing enzyme use relative to a baseline measurement of enzyme use and cost before the enzyme reformulation unit was installed. This removes the risk of buying the enzyme reformulation unit and not using it to it's full potential due to a process slow down or process interruption. The savings sharing calculations result from data captured by the enzyme reformulation unit, including the concentration of enzyme that is to be reformulated, the amount of concentrated enzyme that is reformulated using the enzyme reformulation unit, the amount of chemical reformulant used in the process, and the rate of addition of reformulated enzyme solution to the reactor. The concentration, and the amount of concentrated enzyme and the rate at which the reformulated enzyme solution is added to the reactor provides the total amount of concentrated enzyme used over a certain period of time. This concentrated enzyme usage data is compared to the concentrated enzyme usage data before the present invention was employed. This difference between the usage is the savings to the enzyme user.

The data outlined above can be accessed remotely, or sent by e-mail, ftp or some other networking protocol to the enzyme reformulation unit lessor for calculation of the lease fees due over a specified time period.

Terms used;

JUST-IN-TIME BASIS: The rate at which a commercial enzyme formulation can be reformulated such that a suitable amount of reformulated enzyme is available to continually feed reformulated enzyme to a bioreactor at the rate required to achieve the desired chemical reactions within the bioreactor, while at the same time minimizing the amount of time the reformulated enzyme is stored to minimize bacterial and fungal growth.

REFORMULATING COMMERCIAL ENZYME: Changing the concentration, specific gravity and/or activity of a commercial enzyme preparation by mixing same with aqueous buffers and/or alcohol ethoxylates and/or polymeric compounds such as glycerol, polyethylene glycol, propylene glycol. An example of reformulating enzyme can be found in patent application WO/2010/045168, the complete disclosure of which is incorporated herein by reference.

CHEMICAL REFORMULANTS: Chemicals used to reformulate commercial enzyme such as, but not limited to: aqueous buffers, water, alcohol ethoxylates, polymeric compounds.

### BRIEF DESCRIPTION OF THE DRAWING

FIG. 1 shows a side view of an apparatus for reformulating stabilized enzyme preparations

### DETAILED DESCRIPTION OF THE INVENTION

The present invention relates to a business method for enabling enzyme users to continuously administer enzymes to a reactor wherein the concentration, specific gravity and/or activity of a concentrated enzyme solution is reduced prior to adding said enzyme to the bioreactor. The present invention includes a method of sharing the savings that result when an industrial enzyme user employs the present invention to reduce the amount of enzyme used in the production process. An apparatus to achieve this business method for the administration of enzymes to a reactor is described in patent application WO/2010/045168.

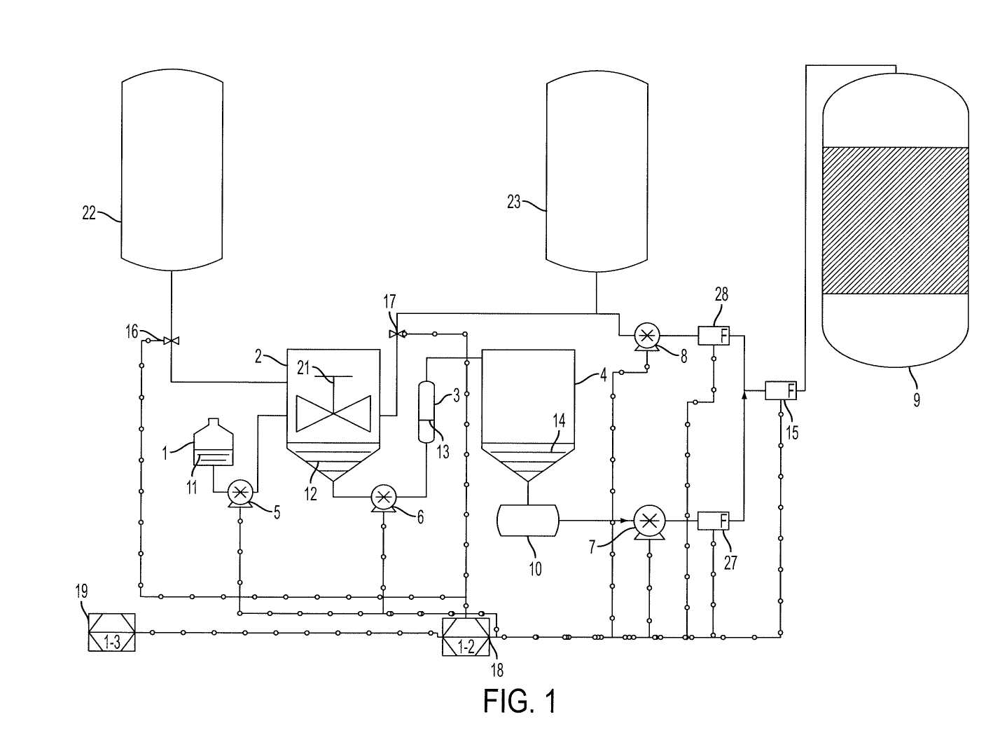

The invention will now be explained with reference to the attached FIG. 1 from WO/2010/045168 without being limited thereto.

As shown in the drawing, the enzyme reformulation apparatus comprises an optional buffer vessel 1, a mixing vessel 2, an optional column 3 containing a metal or metal-impregnated particulate matter 13, a storage vessel 4, an optional surge tank 10. The mixing vessel 2, the storage vessel 4, and surge 10 are constructed of 304 or 316 stainless steel but can be constructed of any desired material suitable to hold the solutions.

The buffer vessel 1 contains a polymeric compound or a mixture of water and polymeric compound or water. The selected compound 11 can be pumped using a variable speed pump 5 to the mixing vessel 2 containing the necessary quantity of water 22 to obtain the desired final concentration of polymeric compound if used. Once the final concentration of buffer is reached in mixing vessel 2, commercial enzyme preparation 23 is added to mixing vessel 2. Optionally the mixture 12 of polymeric compound 11, water 22 and commercial stabilized enzyme preparation 23 can be mixed for between 0.5 minutes and 10 minutes, preferably between 2 minutes and 5 minutes with a stainless steel impeller 21. Any desired mixing device may be used in place of the impeller 21 as desired.

Commercial enzyme preparation 23 is reformulated in the mixing vessel. The dilute polymeric compound is advantageous in that it reduces the concentration of the polymeric stabilizers and other preservatives in which the enzyme is contained, however some stability is still imparted to the reformulated enzyme solution to reduce fouling and bacteria accumulation between the time the commercial enzyme solution is reformulated and the time that it is pumped to the bioreactor.

The reformulation ratio depends on the concentration of enzyme in the commercial enzyme preparation. Currently, concentrations of enzyme used in commercial enzyme preparations for the fuel ethanol, high fructose corn syrup and other industrial applications range from approximately 1% to 20% enzyme. In the future, higher concentrations of enzymes in commercial enzyme preparations may be used. As these concentrations increase, so too will the reformulation ratio. For example, a commercial enzyme preparation with a 75% enzyme concentration may enable a reformulation ration where 250 parts polymeric compound and water are mixed with 1 part commercial enzyme preparation.

In a preferred embodiment, the mixture of polymeric compound 11 and commercial enzyme preparation 23 can be metered, using variable speed pump 6. An optional column 3 is shown. The diluted enzyme solution 14 can be collected in storage vessel 4. An optional surge tank 10 can be connected to the storage vessel 4 so that the storage vessel 4 can be emptied as desired. Depending on the rate at which the enzyme is diluted and the rate at which the diluted enzyme solution is added to the bioreactor 9, diluted enzyme solution may sit in the storage vessel 4 for up to 100 hours.

Diluted enzyme solution can be pumped to the bioreactor or to the pipe that delivers substrate to the bioreactor 24 with a variable speed pump 7. The diluted enzyme solution 14 can be sent to the bioreactor 9 alone or in combination with the commercial stabilized enzyme preparation 23.

Optionally, two variable drive pumps 7 and 8 are in communication with each other and with flowmeters 27, 28 and 15 to ensure delivery of adequate amount of enzyme to the bioreactor 9. For example, if there is a problem with variable drive pump 7, then the flowmeter 27 would communicate to the control system 18 the extent to which flow from pump 7 had slowed. Control system 18 then instructs variable drive pump 8 to take over to an extent that compensates for the decrease in flow from pump 7. Flowmeter 15 ensures that an adequate quantity of enzyme, either reformulated or non-reformulated, is continuously delivered to bioreactor 9. The apparatus is designed such that a stabilized commercial enzyme preparation can be supplied to said apparatus by a valve 17 and supply is independent of the variable drive pump 8. If there is a problem with variable drive 8, commercial stabilized enzyme can be delivered to the apparatus to continue reformulating enzyme and delivering it to bioreactor 9.

The control system 18 for the apparatus contains programmed settings for automated control of all valves and pumps associated with the apparatus and process. A computer screen provides visual cues to operators for tasks to complete such as changing metal or metal-impregnated particulate matter 13 in the column 3 and cleaning the storage tank 4.

In another embodiment of the present invention, the diluted enzyme solution 14 is pumped directly into a bioreactor, without being stored in a storage vessel 4, as in a continuous process.

In another embodiment of the present invention, the diluted enzyme solution 14 is pumped into the substrate-containing pipe 24 that delivers said substrate to the bioreactor. The substrate-containing pipe is preferably between 5 and 12 inches in diameter, more preferably between 6 and 10 inches in diameter. The flow rate of the substrate in said pipe is preferably between 200 and 2000 gallons per minute, more preferably between 400 and 1500 gallons per minute. The pipe through which enzyme flows 25 is preferably between ¼ inch and 2 inches in diameter, more preferably between ½ and 1 inch in diameter.

In another embodiment of the present invention, the substrate slurry consists of between 10 and 40% solids where the solids consist of protein, carbohydrate, fiber, and/or fat.

In another embodiment of the present invention, the polymeric compound and water mixture are mixed with stabilized enzyme preparation 23 in-line, using an in-line mixer and pumped directly to the bioreactor, without being mixed in a mixing vessel 2 and without being stored in a storage vessel 4.

In another embodiment of the present invention, control system 18 is in communication with a central control system 19 that monitors the entire production facility.

The invention will be further explained by the following non-limiting examples.

### Example 1

A fuel ethanol plant purchases glucoamylase, an enzyme that hydrolyses maltodextrins, from a commercial enzyme supplier. Glucoamylase (70 Gallons) is dosed into a 510,000 Gallon fermenter over 6 hours, (rate=735 mL/min) to hydrolyse maltodextrin from corn and/or sorghum to glucose. Yeast in the fermenter metabolize the glucose, one of the by-products of said metabolism being ethanol. In subsequent production steps the ethanol is distilled and concentrated to produce fuel ethanol. Glucoamylase that has a high activity can produce glucose in the fermenter too quickly for the yeast to metabolize said glucose efficiently. The result is a fermenter containing high concentrations of glycogen and lower than optimal concentrations of ethanol. As a result, commercial enzyme producers formulate glucoamylase solutions that have relatively low activity. These enzymes are shipped, usually via transport trailers, from central enzyme production locations. In many cases, the enzyme must be shipped over great distances making transport costly, time-consuming and causing greenhouse gas and volatile compound emissions from the burning of fossil fuel. Currently, shipments of glucoamylase are received by the fuel ethanol plant every 38 days and are stored in a large stainless steel tank. The activity of the glucoamylase is between 900 and 1000 amyloglucosidase Units/gram (AGU/g) where 1 AGU is defined as the amount of enzyme that cleaves 1 umol of maltose per minute under standard assay conditions. The stability of the glucoamylase is up to 1 year.

As an example of the present invention, a glucoamylase enzyme formulation with activity of 4000 to 6000 AGU/g can be obtained. Prior to dosing the concentrated glucoamylase into the fermenter, the glucoamylase was reformulated using a device such as the one specified in patent application WO/2010/045168. The glucoamylase activity was reduced to 1000 AGU/g by adding 4 volumes of a 10% (v/v) solution of propylene glycol in water. The water is obtained from a reverse osmosis unit. The glucoamylase enzyme, by virtue of it's increased activity, will be drawn from the storage tank at a slower rate than the case where the enzyme activity is 1000 AGU/g. As a result, the enzyme in the storage tank will last 5 times longer. Enzyme shipments can now be received every 190 days. Instead of receiving approximately 10 shipments/year, the plant can now receive less than 2 shipments per year, reducing the cost of shipping significantly as well as reducing the harmful emissions that result from multiple, and now unnecessary shipments. The reduction in emissions will reduce taxes payable in the event the biorefinery is subject to a carbon tax, and will reduce the number of emission credits required to offset greenhouse gas emissions in the event the ethanol producer is subject to a cap & trade emissions reduction policy. These offset credits can also be sold in public carbon markets when the enzyme manufacturing company or the biorefinery is subject to a cap & trade emissions reduction scheme that permits the use of offset credits to offset greenhouse gas emissions.

This reduction in activity can be customized for each plant using a device as described in patent application WO/2010/045168. Therefore, while currently enzyme users are bound by the concentrations provided by enzyme suppliers, the present invention, coupled with the device presented in WO/2010/045168, provide enzyme users the flexibility to customize the enzyme concentration, activity and/or specific gravity to meet the needs of their unique plant and their unique operating conditions. Modifying enzyme concentration, activity, and/or specific gravity can only be performed on-site and just-in-time because the reduction in concentration, activity and/or specific gravity also reduces the concentration of stabilizers that are included in the commercial formulation for the purpose of storing the enzyme for up to 1 year. A reduction in stabilizer concentration to the extent listed above reduces the stability of the enzyme to between 12 and 48 hours.

It is well known in the art that enzyme formulations can be concentrated in a way that increases the activity per unit volume. This has been done with commercial alpha-amylase enzymes, the benefit being a reduction in shipping costs. However, as enzyme activity per unit volume increases, the concentration and specific gravity of the enzyme formulation often increase. Enzyme formulations with high specific gravity and/or high concentration can be difficult to pump at accurate rates with industrial pumps.

A solution to this problem is to change the concentration, specific gravity and/or activity after delivery of the enzyme concentrate. By reformulating the enzyme concentrate on-site using a unit such as the one described in patent application WO/2010/045168, the enzyme activity, concentration and/or specific gravity can be customized for the specific application. Moreover, the enzyme activity, concentration and/or specific gravity can be changed as the substrate concentration changes and as the substrate source changes.

1. A method for administering enzymes to a reactor on a just-in-time basis comprising:
reformulating an enzyme concentrate to produce a reformulated enzyme solution of a desired concentration, specific gravity and/or activity, before addition of said enzyme to a bioreactor, at a rate that allows continuous delivery of said reformulated enzyme solution at the rate required to effect a desired enzyme-catalyzed reaction.

2. A method for administering enzymes to a reactor according to claim 1, wherein enzymes are added to the reactor continuously.

3. A method for administering enzymes to a reactor according to claim 1, wherein enzymes are added through a pipe ¼ inch in diameter to a substrate flowing at between 200 and 2000 Gallons/minute

4. A method according to claim 3, wherein the substrate is a slurry containing between 10 and 40% solids

5. A method for administering enzymes to a reactor on a just-in-time basis comprising:
reformulating an enzyme concentrate to the desired concentration, specific gravity and/or activity at a rate that allows continuous delivery of said reformulated enzyme at the rate required to effect a desired enzyme-catalyzed reaction;
recording the data describing the degree to which the enzyme concentration, specific gravity and/or activity changes over time;
storing said data in a database and/or data table; and
sending said data to a server over a network.

6. A method for administering enzymes to a reactor according to claim 3, wherein the recorded information includes the quantities of chemical reformulants used to reformulate the enzyme concentrate.

7. A method for administering enzymes to a reactor according to claim 3, wherein the recorded information includes the flow rate of reformulated enzyme to the bioreactor.

8. A method for accurately administering enzymes to a reactor comprising;
reformulating a commercial enzyme concentrate, on a just-in-time basis, for addition to a bioreactor using an enzyme reformulation system capable of reformulating commercial enzyme concentrate in a biorefinery or chemical plant on a just-in-time basis;
using said enzyme reformulation system to reformulate enzyme concentrate to a concentration, specific gravity and/or activity suitable to the biorefinery or chemical process;
reformulating enzyme concentrate by a method that prevents bacterial contamination;
recording enzyme reformulation metrics over time; and
billing said biorefinery or chemical plant for the amount of enzyme reduced by reformulation.

9. A method for reducing greenhouse gas emissions during the transport of enzymes from an enzyme manufacturing facility to a biorefinery or chemical plant comprising:
reformulating a commercial enzyme concentrate on a just-in-time basis, for addition to a bioreactor using an enzyme reformulation system capable of reformulating commercial enzyme concentrate in a biorefinery or chemical plant; and
ordering enzyme for delivery less frequently than under conditions where a commercial enzyme concentrate is not reformulated on a just-in-time basis.

10. A method for reducing greenhouse gas emissions according to claim 9, wherein the reduction in greenhouse gas emissions from less frequent delivery of enzymes from the enzyme manufacturing facility to a biorefinery or chemical plant is calculated as a reduction of the biorefinery or chemical plant's total emissions.

11. A method for reducing greenhouse gas emissions according to claim 10 wherein the biorefinery or chemical plant uses the reduction in greenhouse gas emissions from less frequent delivery of enzymes from the enzyme manufacturing facility to a biorefinery or chemical plant to generate cap & trade offset credits to offset greenhouse gas emissions elsewhere in the biorefinery or chemical plant.

12. A method for reducing greenhouse gas emissions according to claim 11 wherein the biorefinery or chemical plant sells cap & trade offset credits in a public carbon market.

13. A method for reducing greenhouse gas emissions according to claim 9, wherein the reduction in greenhouse gas emissions from less frequent delivery of enzymes from the enzyme manufacturing facility to a biorefinery or chemical plant is calculated as a reduction of the enzyme manufacturing plant's total emissions.

14. A method for reducing greenhouse gas emissions according to claim 13, wherein the enzyme manufacturing plant uses the reduction in greenhouse gas emissions from less frequent delivery of enzymes from the enzyme manufacturing facility to a biorefinery or chemical plant to generate cap & trade offset credits to offset greenhouse gas emissions elsewhere in the enzyme manufacturing plant.

15. A method for reducing greenhouse gas emissions according to claim 14, wherein the enzyme manufacturing plant sells cap & trade offset credits in a public carbon market

16. A method for administering enzymes to a reactor on a just-in-time basis comprising:
reformulating a plurality of enzyme concentrates to produce a reformulated enzyme mixture wherein each enzyme component is a desired concentration, specific gravity and/or activity, before addition of said enzyme mixture to a bioreactor, at a rate that allows continuous delivery of said reformulated enzyme mixture at the rate required to effect a desired enzyme-catalyzed reaction.

17. A method for administering enzymes to a reactor according to claim 16, wherein the enzyme mixture is added to the reactor continuously.

18. A method for administering enzymes to a reactor according to claim 16, wherein enzymes are added through a pipe ¼ inch in diameter to a substrate flowing at between 200 and 2000 Gallons/minute

19. A method according to claim 18, wherein the substrate is a slurry containing between 10 and 40% solids
